(19) United States
(12) Patent Application Publication
(10) Pub. No.: US 2015/0141998 A1
(43) Pub. Date: May 21, 2015

(54) SYSTEM AND METHOD FOR FORMING A CURVED TUNNEL IN BONE

(71) Applicants: Ali Kiapour, PROVIDENCE, RI (US); JOSE FERNANDEZ, WRENTHAM, MA (US)

(72) Inventors: Ali Kiapour, PROVIDENCE, RI (US); JOSE FERNANDEZ, WRENTHAM, MA (US)

(73) Assignee: SYMMETRY MEDICAL MANUFACTURING, INC, WARSAW, IN (US)

(21) Appl. No.: 14/231,894

(22) Filed: Apr. 1, 2014

Related U.S. Application Data
(63) Continuation-in-part of application No. 14/084,460, filed on Nov. 19, 2013.

(51) Int. Cl. A61B 17/16

(52) U.S. Cl. CPC A61B 17/1642
USPC 606/80

(57) ABSTRACT

A drill system for forming a curved tunnel in a bone includes a drill bit guidance device. The drill bit guidance device includes an elongated stationary outer tube extending along a first axis and an elongated inner tube assembly. The elongated inner tube assembly is configured to slidably move within the elongated stationary outer tube along the first axis and to exit from a distal end of the elongated outer tube. The elongated inner tube assembly includes an actuating tube and an actuator tube. The actuating tube slidably moves within the actuator tube and includes a plurality of semi-cross-sectional slots extending in a slot direction perpendicular to the first axis. Each slot of the actuating tube is configured to collapse inward in the slot direction when exiting the distal end of the elongated outer tube, thereby causing a distal end portion of the elongated inner tube assembly to follow a curved path.

### CROSS REFERENCE TO RELATED CO-PENDING APPLICATIONS

This application is a continuation-in-part and claims the benefit of U.S. application Ser. No. 14/084,460 filed on Nov. 19, 2013 and entitled SYSTEM AND METHOD FOR FORMING A CURVED TUNNEL IN BONE, which is commonly assigned and the contents of which are expressly incorporated herein by reference.

### FIELD OF THE INVENTION

The present invention relates to a system and a method for forming a curved tunnel in a bone and in particular to device that uses a tube with a slotted front end to form a U-shaped tunnel in the bone.

### BACKGROUND OF THE INVENTION

In several surgical procedures opening of curved tunnels in bone is needed. Examples of these type of surgical procedures include rotator cuff repair and Achilles tendon repair where re-attaching of muscle and ligaments to bone is performed.

Prior art technologies for performing these type of surgical procedures involve drilling two separate openings that have intersecting screw paths in order to form the attachment point. These prior art procedures are invasive because they require drilling two separate intersecting openings from two different directions. There is also the possibility that the two drilled openings do not intersect due to miscalculations of the drill path or inaccuracy in the drilling process. This may result in drilling additional openings in the bone, which increases the level of complexity of the procedure, increases the operation time and also increases the bleeding risk of the patient.

Accordingly, systems and methods that simplify the above mentioned surgical procedures are desirable.

### SUMMARY OF THE INVENTION

The present invention provides a system and a method for forming a curved tunnel in a bone by using a drill bit guidance device. The drill bit guidance device includes a tube with a slotted front end, whereby the slots collapse and cause the tube to curve at a predetermined radius as the drill bit moves forward.

The present invention also provides a system and a method for attaching a ligament to a bone by forming a curved tunnel in a bone. The method includes first providing a drill and a drill bit guidance device that guides the drill bit to form a U-shaped curved tunnel. Next, drilling a U-shaped tunnel into a bone by entering into a first bone location with the guided drill bit and exiting from a second bone location. Next, threading a suture through the opened U-shaped tunnel and through the ligament that needs to be attached to the bone, and then attaching the ligament to the bone with the threaded suture.

In general, in one aspect, the invention features drill system for forming a curved tunnel in a bone including a drill bit guidance device. The drill bit guidance device includes an elongated stationary outer tube extending along a first axis and an elongated inner tube assembly. The elongated inner tube assembly is configured to slidably move within the elongated stationary outer tube along the first axis and to exit from a distal end of the elongated outer tube. The elongated inner tube assembly includes an actuating tube and an actuator tube. The actuating tube is configured to slidably move within the actuator tube and comprises a plurality of semi-cross-sectional slots extending in a slot direction perpendicular to the first axis. Each slot of the actuating tube is configured to collapse inward in the slot direction when exiting the distal end of the elongated outer tube, thereby causing a distal end portion of the elongated inner tube assembly to follow a curved path.

Implementations of this aspect of the invention may include one or more of the following features. A distal end of the actuator tube is connected to a distal end of the actuating tube. The actuator tube comprises a partially slotted distal end portion and the slots of the actuating tube are displaced by 180 degrees relative to slots of the actuator tube. The drill bit guidance device further includes a handle, a thrust assembly, and a cam assembly. The handle includes a first through-opening dimensioned to receive and hold the thrust assembly, the cam assembly and the elongated stationary outer tube. The thrust assembly includes a thrust shaft, and the thrust shaft is configured to slide within the first through-opening of the handle. The thrust assembly further includes a shank chuck, a thrust end cap, and first and second thrust bushings. The drill bit guidance device further includes a drive shaft, and a drill bit. The actuating tube surrounds the drive shaft and the drill bit is attached to a distal end of the drive shaft and a proximal end of the drive shaft is attached to the thrust shaft. Proximal ends of the actuating tube, the drive shaft and the actuator tube are linked together via the cam assembly, and the cam assembly is configured to maintain constant the ratio of the length of the drive shaft to the length of the collapsed slotted distal end portion of the actuating tube, thereby maintaining constant a radius of the curved path. The cam assembly includes a central cam and a distal cam and the central cam and distal cam are configured to turn and slide within the first through-opening of the handle. The radius of the curved path is configured to remain constant by maintaining constant the ratio of the length of the flexible drive cable to the length of the collapsed slotted distal end portion of the elongated actuating tube. The drive shaft includes a flexible cable and the flexible cable comprises Nitinol wire or stainless steel wire. Each of the slots in the actuating tube is wider at the bottom than at the top. The drill system further includes a drill driver comprising a front opening configured to receive removable attachments, and the drill bit guidance device is configured to be removably attached to the front opening of the drill driver.

Among the advantages of this invention may be one or more of the following. The invention provides a method for attaching a ligament to a bone without the use of screws. This eliminates the need for introducing screws or other foreign objects in the bone, which in turn reduces the risks of infection and rejection of the foreign object. The method opens a single curved path, rather than opening at least two separate paths from two different directions and then trying to orient them so that they intersect each other. This reduces the complexity of the surgical procedure, reduces the operation time and reduces the risk of prolonged bleeding. Furthermore, there no need for a separate device for pulling a suture through the intersecting straight paths.

The details of one or more embodiments of the invention are set forth in the accompanying drawings and description bellow. Other features, objects and advantages of the invention will be apparent from the following description of the preferred embodiments, the drawings and from the claims.

### BRIEF DESCRIPTION OF THE DRAWINGS

Referring to the figures, wherein like numerals represent like parts throughout the several views:

FIG. 1A-FIG. 1E depict schematic views of the steps for performing a ligament re-attachment procedure according to this invention;

FIG. 1F is a process diagram of a ligament re-attachment procedure according to this invention;

FIG. 2A is a perspective view of a drill bit guidance device according to this invention;

FIG. 2B is a perspective view of the drill bit guidance device of FIG. 2A with the slotted tube in the “straight” position;

FIG. 2C is a perspective view of the drill bit guidance device of FIG. 2A with the slotted tube in the “curved” position;

FIG. 3 is a partially exploded view of the drill bit guidance device of FIG. 2A;

FIG. 4 is an exploded view of the distal end of the drill bit guidance device of FIG. 2A;

FIG. 5A is a transparent view of the distal end of the drill bit guidance device of FIG. 2A;

FIG. 5B depicts a partially exploded view of the distal end of the drill bit guidance device of FIG. 2A with the slotted tube in the “straight” position;

FIG. 5C depicts the distal end of the drill bit guidance device of FIG. 2A with the slotted tube in the “straight” position;

FIG. 5D depicts the distal end of the drill bit guidance device of FIG. 2A with the slotted tube in the “curved” position;

FIG. 6A depicts a side transparent view of the drill bit guidance device of FIG. 2A with the slotted tube in the “straight” position;

FIG. 6B depicts a side transparent view of the drill bit guidance device of FIG. 2A with the slotted tube in the “curved” position;

FIG. 7A depicts an enlarged side view of the thrust assembly of the drill bit guidance device of FIG. 2A with the slotted tube in the “curved” position;

FIG. 7B depicts an enlarged side view of the thrust assembly of the drill bit guidance device of FIG. 2A with the slotted tube in the “straight” position;

FIG. 8A depicts components of the proximal end of the drill bit guidance device of FIG. 2A; and

FIG. 8B depicts exploded components of the proximal end of the drill bit guidance device of FIG. 2A.

### DETAILED DESCRIPTION OF THE INVENTION

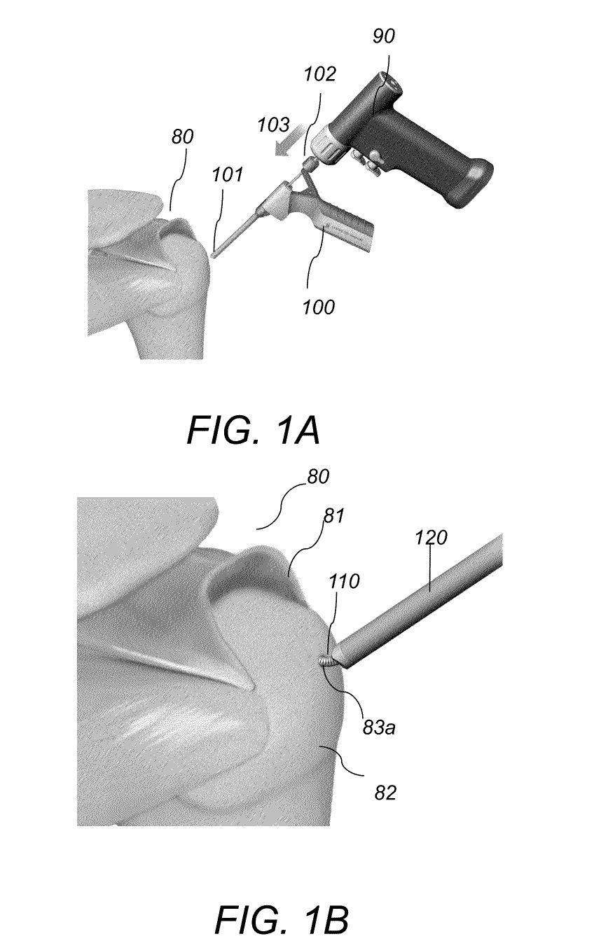

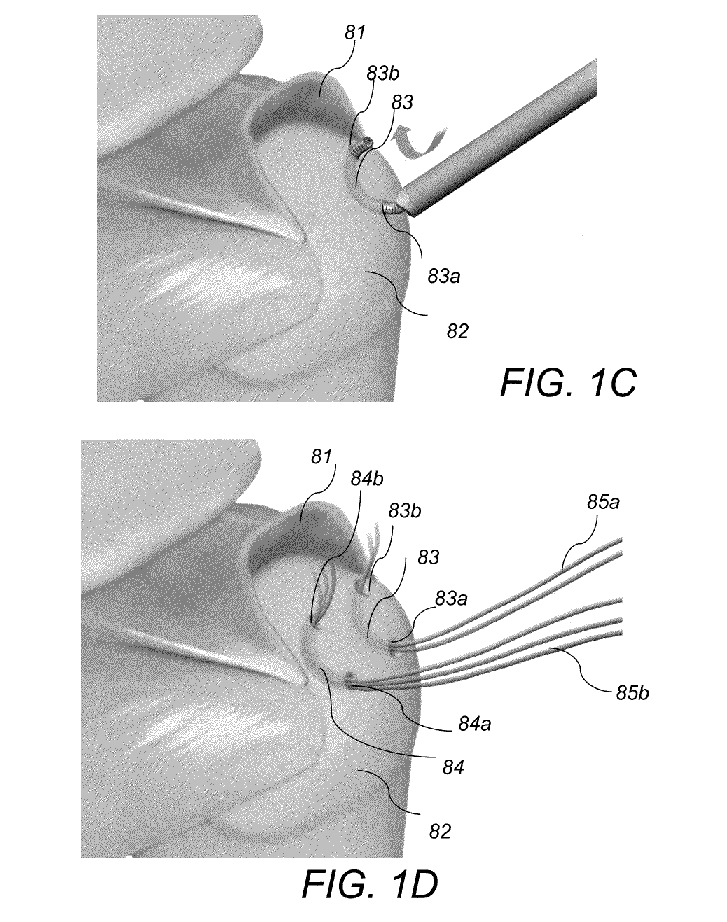

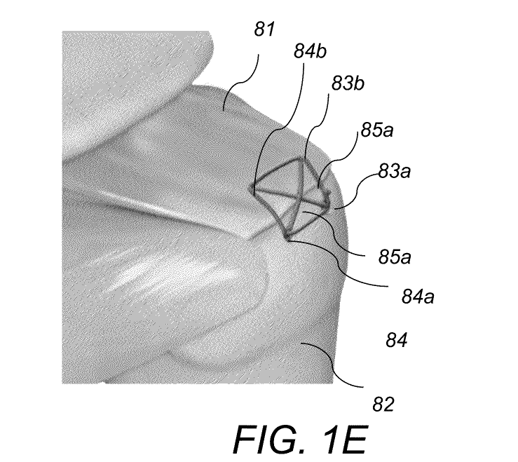

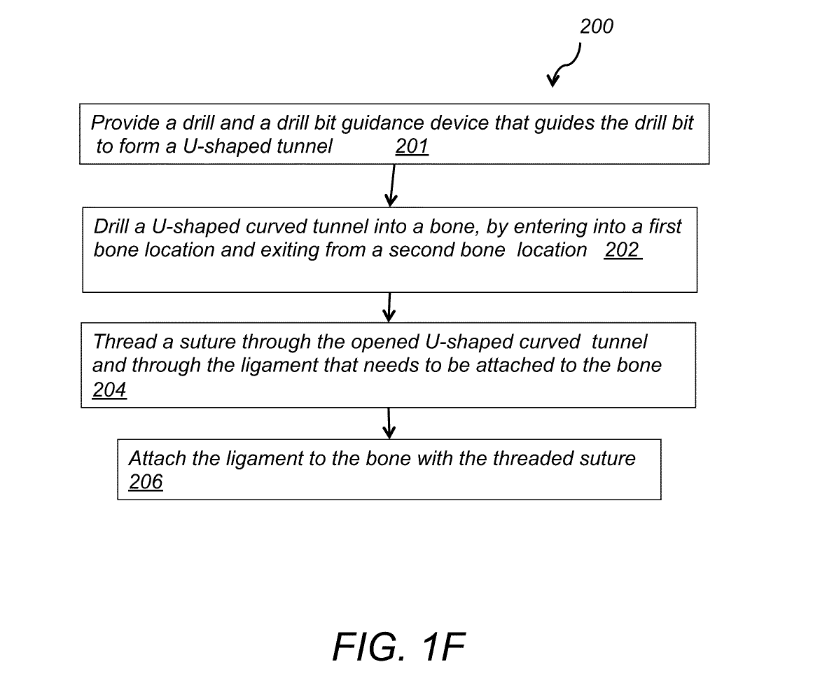

Referring to FIG. 1A-FIG. 1E, a ligament re-attachment surgical procedure in the rotator cuff area 80 includes the following. First, a drill 90 and a drill bit guidance device 100 are provided. Drill bit guidance device 100 is placed in front of drill 90 and includes a mechanism that guides the drill bit to drill a U-shaped curved tunnel. Next, the surgeon creates access to the bone area 82, and holds the distal end 101 of device 100 firmly against the bone 82 with one hand, while the other hand holds the power drill 90 that is attached to the proximal end 102 of the device 100. Next, the surgeon operates the drill 90 so that it rotates and he pushes the drill forward in the direction indicated by arrow 103 toward the distal end 101 of device 100, as shown in FIG. 1A. During this process, a drill bit or burr feeds through the distal end 101 of device 100 and enters a first bone location 83a. As the drill bit feeds out, it is guided by a tube 110 that causes the drill bit to move through a curved path, as shown in FIG. 1C. The curved path has a predetermined radius and forms a U-shaped tunnel 83. The drilling process continues until the drill bit exits the bone from a second bone location 83b. Next, while still holding the device 100 in place, the surgeon retracts the power drill 90 leaving behind an open U-shaped curved tunnel 83. The process is repeated again and a second U-shaped tunnel 84 is opened. Next, sutures 85a, 85c are threaded through the opened U-shaped tunnels 83, 84, respectively, and through the ligament 81 that needs to be attached to the bone 82, as shown in FIG. 1D. Finally, the sutures 85a, 85b are tied and ligament 81 is attached to the bone 82, as shown in FIG. 1E.

Referring to FIG. 1F, the process diagram 200, for attaching a ligament to a bone includes the following. First, providing a drill and a drill bit guidance device that guides the drill bit to form a U-shaped tunnel (201). Next, drilling a U-shaped tunnel into a bone by entering into a first bone location with the guided drill bit and exiting from a second bone location (202). Next, threading a suture through the opened U-shaped tunnel and through the ligament that needs to be attached to the bone, and then attaching the ligament to the bone with the threaded suture (206).

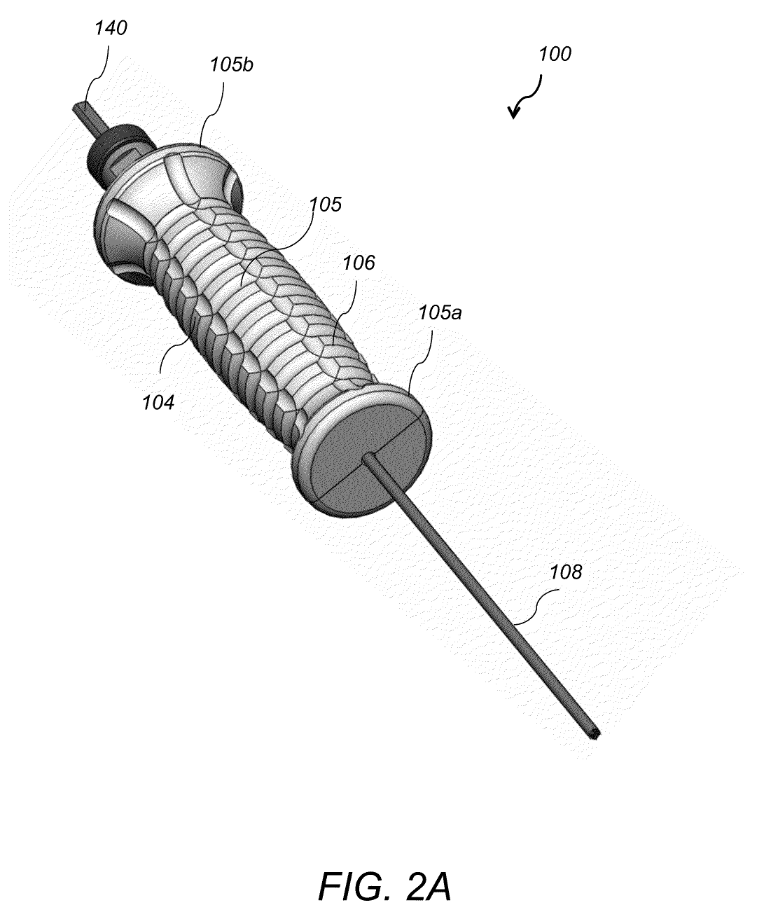

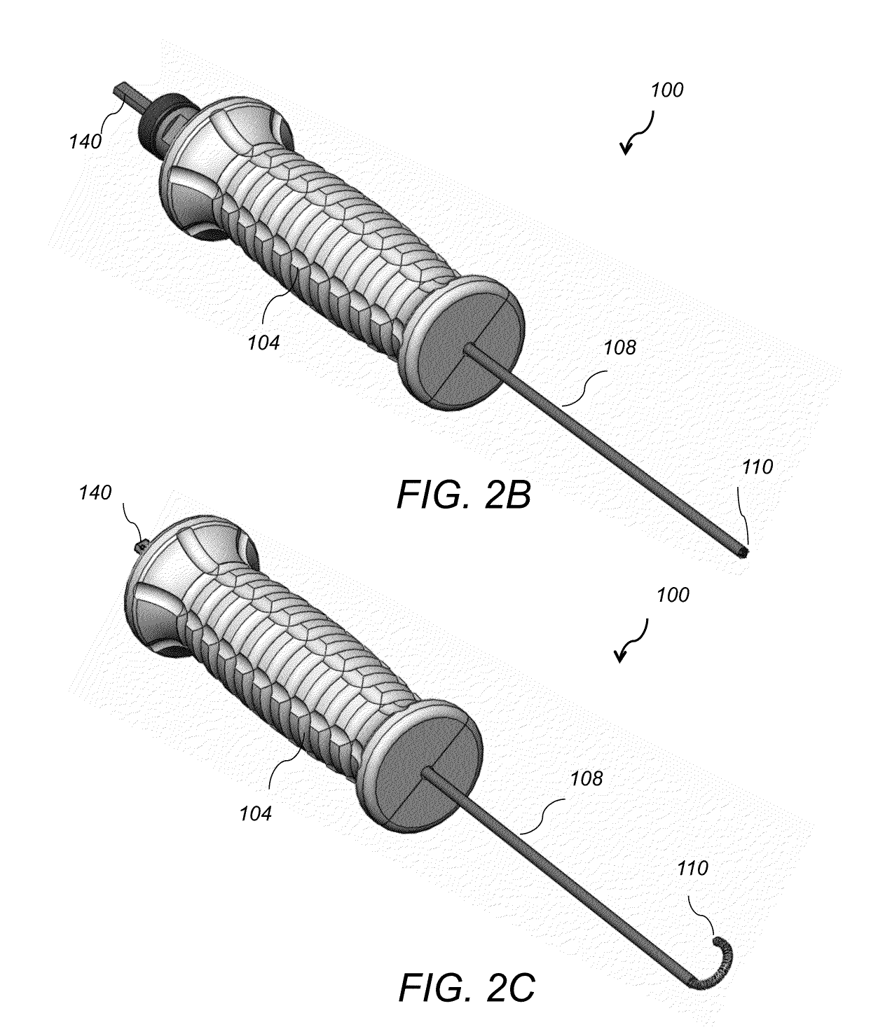

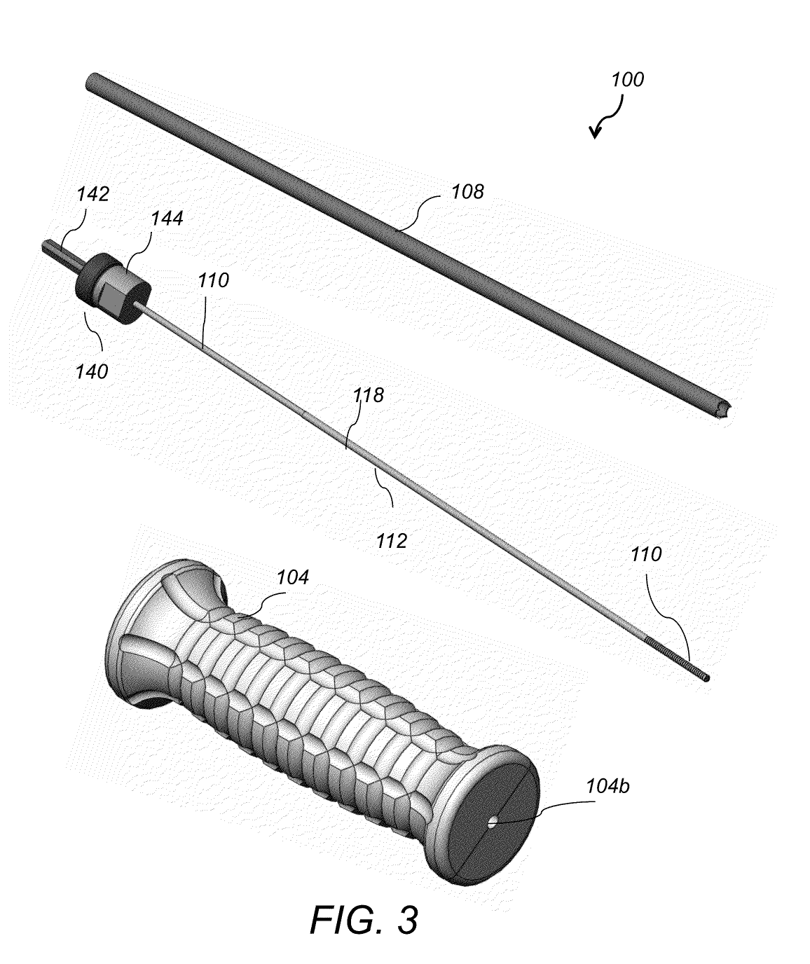

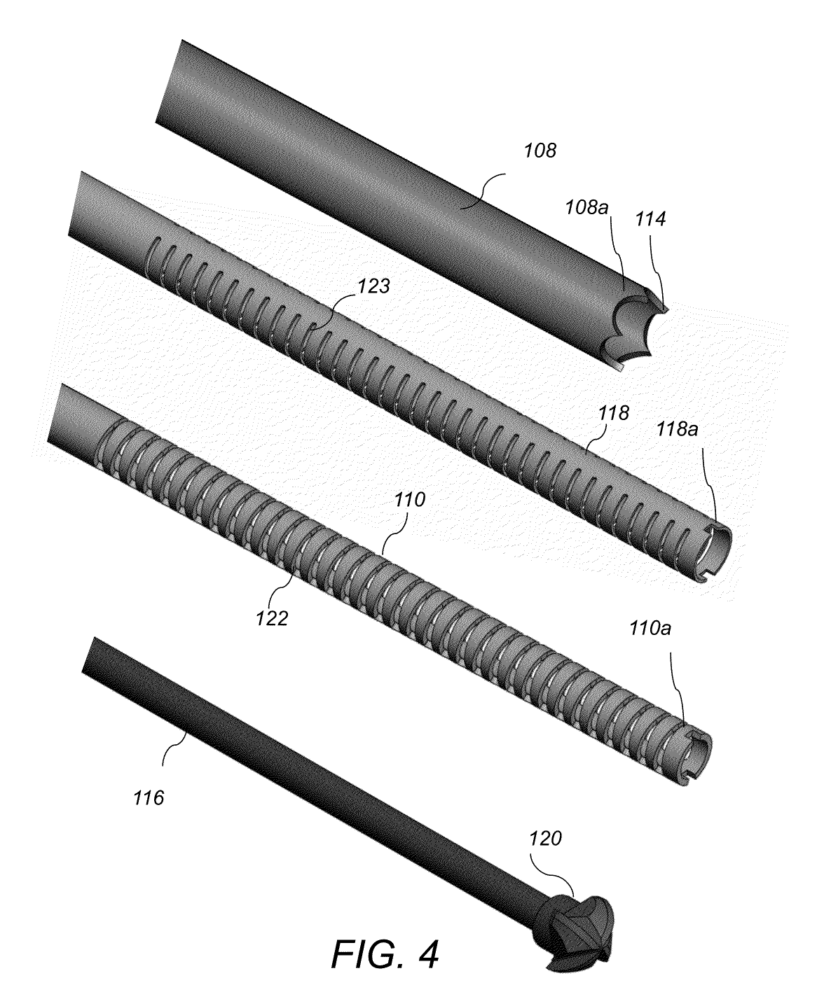

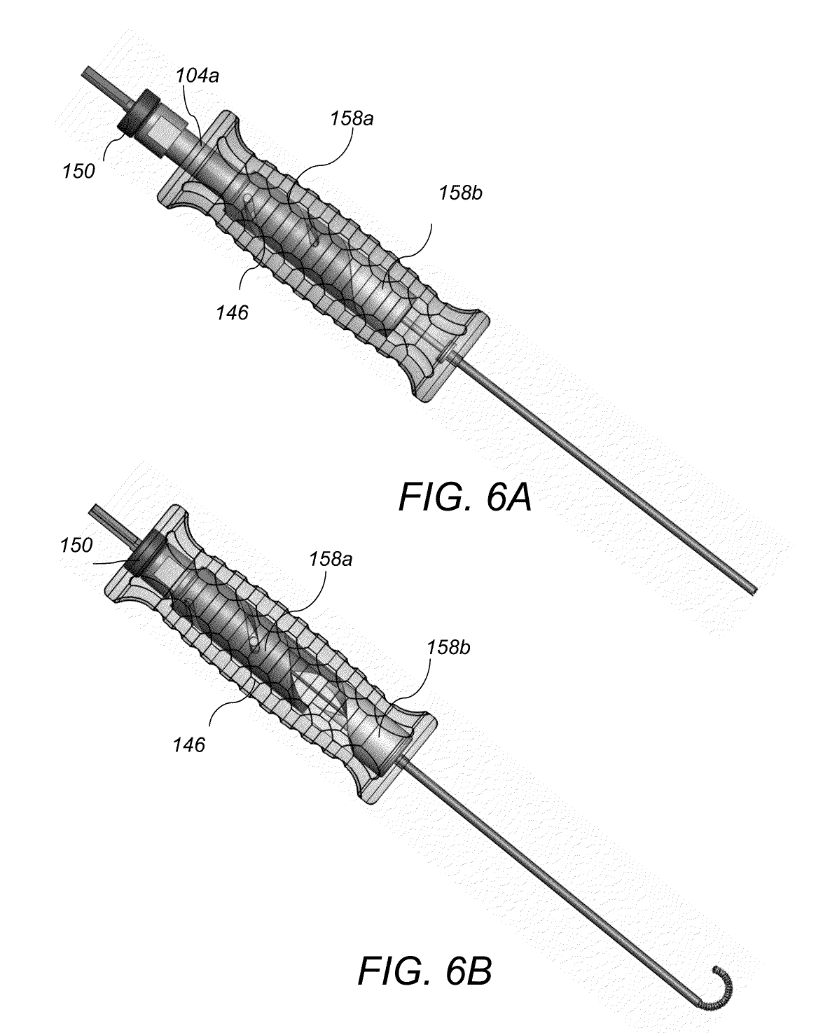

Referring to FIG. 2A, FIG. 3, and FIG. 6A and FIG. 6B, a drill bit guidance device 100 includes a universal grip/handle 104, an outer stationary tube 108, an inner tube assembly 112, a thrust assembly 140, and a cam assembly 146. Thrust assembly 140 transfers the drive motion of a power drill 90 to a drive shaft 116 that is housed within the inner tube assembly 112, as shown in FIG. 4. The universal grip/handle 104 has a cylindrical body 105 and includes flared out distal and proximal ends 105a, 105b, respectively. Handle 104 is used for holding the device 100 with one hand while holding the power drill 90 with the other hand. The outer surface of the cylindrical body 105 includes ridges and protrusions 106 that provide a secure grip. Handle 104 has a first through opening 104a dimensioned to receive and hold the thrust assembly 140 and the cam drive assembly 146, as shown in FIG. 6A. Through-opening 104a terminates into an opening 104b having a diameter smaller than the diameter of opening 104a and dimensioned to hold and support the outer stationary tube 108. Thrust assembly 140 slides within through-opening 104a. Cam assembly 146 is a cylindrical type cam and includes a central cam 158a and a distal cam 158b.

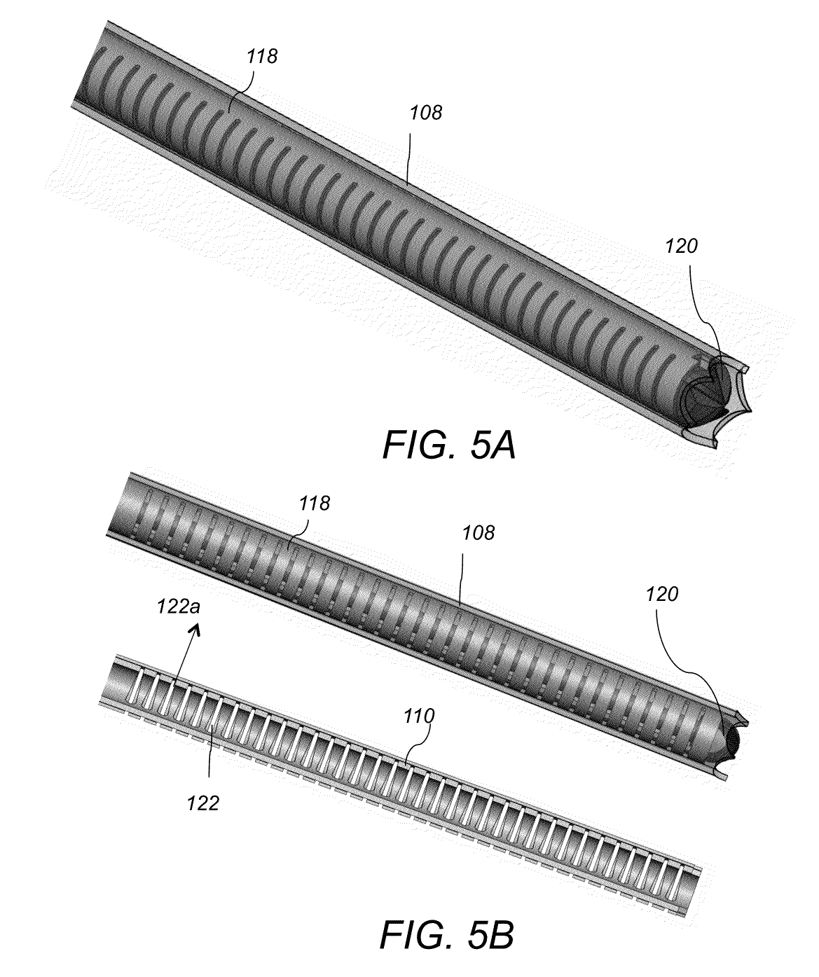

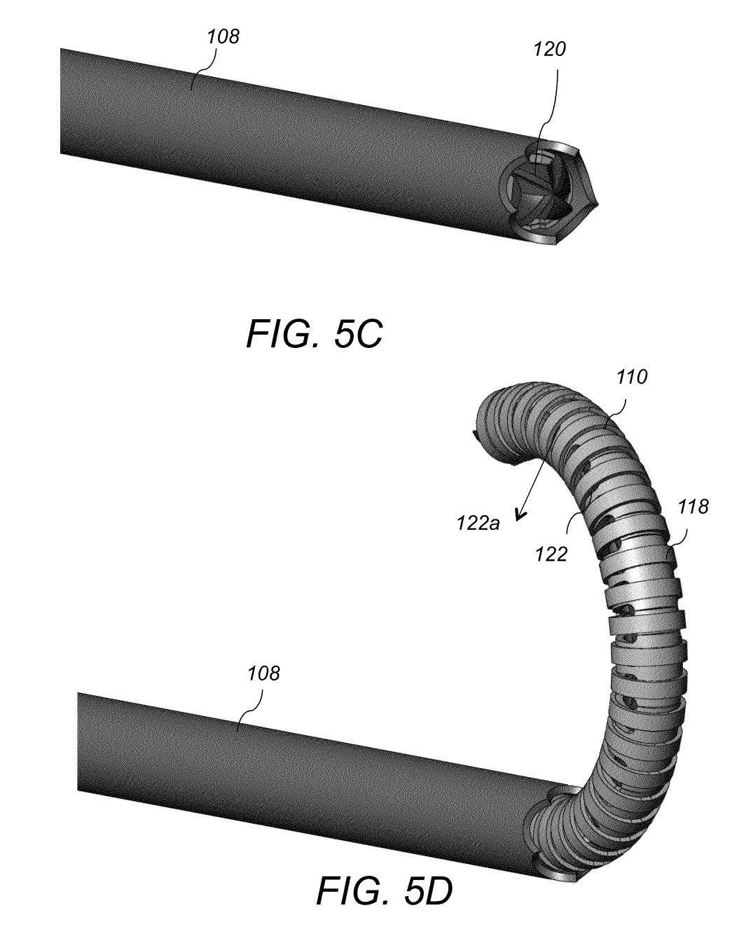

Referring to FIG. 3 and FIG. 4, inner tube assembly 112 is housed within outer tube 108 and includes an outer actuator tube 118 with a partially slotted front portion, an inner actuating tube 110 also with a partially slotted front portion (“slotted tube”), a drive shaft 116, and drill bit 120. Drive shaft 116 is made of a flexible cable and has a distal end attached to the drill bit 120. The flexible cable drive shaft 116 curves with the curving of the slotted tube 110 and allows the drill bit 120 to follow a curved path, as will be described below. In one example, flexible cable 116 is made of Nitinol wire, stainless steel wire or cable. The distal end 118a of the actuator tube 118 is connected to the distal end 110a of the actuating tube 110. The assembled drive shaft/flexible cable 116, and drill bit 120, is dimensioned to be housed and move within the actuating tube 110 and the actuating tube 110 is dimensioned to be housed and move within the actuator tube 118. The actuator tube 118 is dimensioned to be housed and move within the stationary outer tube 108. The distal end 108a of the stationary tube 108 has spikes 114 for securing the tip of the device into the bone during the procedure. The slotted front portion of actuating tube 110 includes slots 122 that are oriented along direction 122a and are slightly wider at the bottom of each slot than at the top of the slot, as shown in FIG. 5B. The slots 123 in the front end of the actuator tube 118 are oriented 180 degrees apart with respect to the orientation 122a of the slots 122 in the front end of the actuating tube 110.

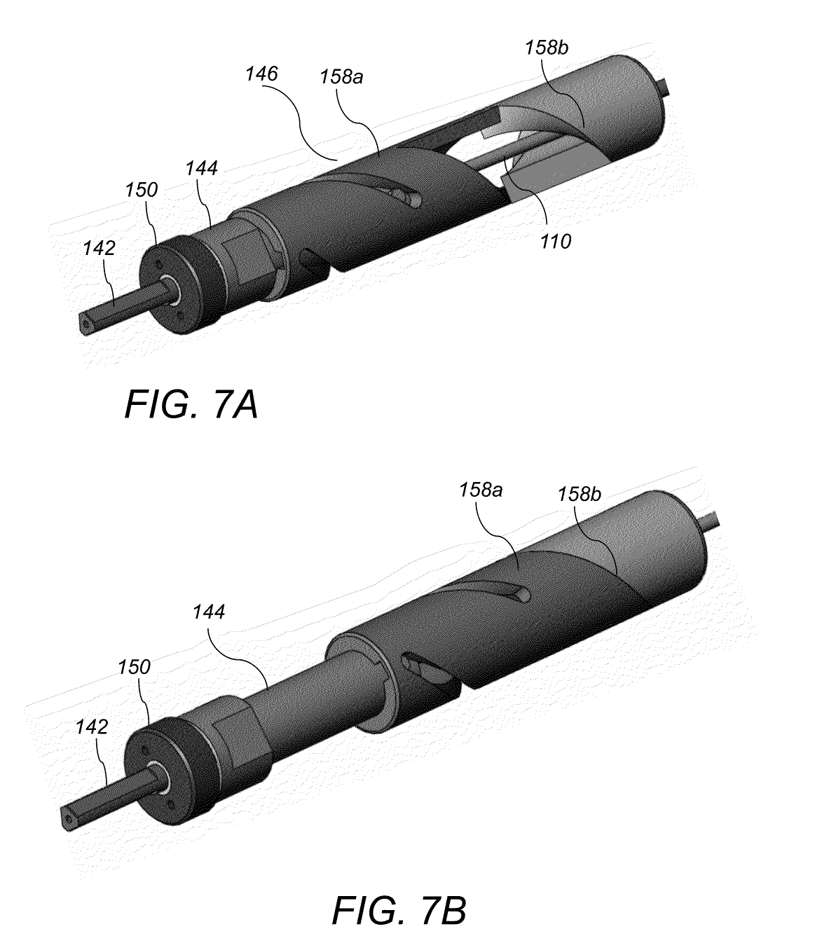

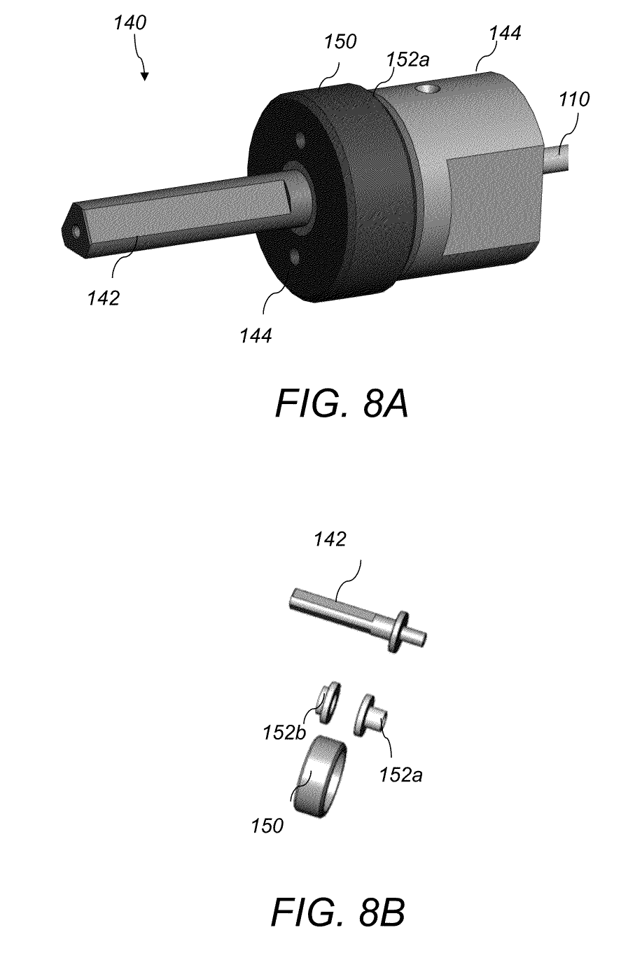

Referring to FIG. 7A, FIG. 7B, FIG. 8A and FIG. 8B, thrust assembly 140 includes a shank chuck 142 that connects to the drive shaft 116, a thrust shaft 144, a thrust end cap 150, and thrust bushings 152a, 152b. Thrust assembly 140 is encapsulated within opening 104a of handle 104 and within the cam assembly 146, as shown in FIG. 6A and FIG. 6B.

In operation, as the drill bit 120 feeds out of the outer tube 108, the slotted front end of actuating tube 110 that is connected to the drill bit 120 follows. As each slot 122 of the actuating tube front exits the outer tube 108, it collapses inward in the slot direction 122a, as shown in FIG. 5D. This inward collapse of the slots 122 causes the length of the actuating tube 110 to become shorter than the length of the flexible cable 116 and this results in curving of the flexible cable 116, which in turn causes the drill bit 120 to follow a curved path as it moves forward. The ratio of the length of the drive shaft 116 to the length of the collapsed slotted tube 110 remains constant throughout the entire range of the drill bit movement causing the radius of the curved path to remain constant. The distal end 118a of the actuator tube 118 is connected to the distal end 110a of the actuating tube 110 and causes each slot 122 to collapse as it exits the outer tube 108. In this embodiment, the proximal end of actuator tube 118 is attached to the distal cam 158b and the proximal end of actuating tube 110 is attached to the distal end of thrust shaft 144. Drill bit 120 and drive shaft 116 move the same amount as the actuating tube 110. The length of the actuating tube 110 is different than the length of the flexible shaft 116 in the curved position. The cam assembly 146 provides the proper feed ratio so that the drill bit 120 moves in a curved path with a predetermined radius. FIG. 2A depicts the drill bit guidance device 100 with the slotted tube assembly in the start position. FIG. 2B depicts the drill bit guidance device 100 with the slotted tube in the “straight” position. FIG. 2C depicts the drill bit guidance device 100 with the slotted tube in the “curved” position. FIG. 5C depicts the distal end of the slotted tube in the “straight” position and FIG. 5D depicts the distal end of the slotted tube in the “curved” position.

FIG. 6A and FIG. 6B depict side transparent side views of the drill bit guidance device 100 with the slotted tubes in the “straight” and “curved” positions, respectively. As the thrust shaft 144 slides into the slots of the central cam 158a, the cam 158a turns resulting in turning of the distal cam 158b. As the distal cam 158b turns it slides forward and thereby provides a different feed rate to the actuator tube 118 than the feed rate of the actuating tube 110.

Several embodiments of the present invention have been described. Nevertheless, it will be understood that various modifications may be made without departing from the spirit and scope of the invention. Accordingly, other embodiments are within the scope of the following claims.

What is claimed is:

1. A drill system for forming a curved tunnel in a bone comprising:
a drill bit guidance device comprising an elongated stationary outer tube extending along a first axis and an elongated inner tube assembly and wherein said elongated inner tube assembly is configured to slidably move within the elongated stationary outer tube along the first axis and to exit from a distal end of the elongated outer tube;
wherein said elongated inner tube assembly comprises an actuating tube and an actuator tube, wherein the actuating tube is configured to slidably move within the actuator tube and comprises a plurality of semi-cross-sectional slots extending in a slot direction perpendicular to the first axis and wherein each slot of the actuating tube is configured to collapse inward in the slot direction when exiting the distal end of the elongated outer tube, thereby causing a distal end portion of the elongated inner tube assembly to follow a curved path.

2. The drill system of claim 1, wherein a distal end of the actuator tube is connected to a distal end of the actuating tube, and wherein the actuator tube comprises a partially slotted distal end portion and wherein the slots of the actuating tube are displaced by 180 degrees relative to slots of the actuator tube.

3. The drill system of claim 1, wherein the drill bit guidance device further comprises a handle, a thrust assembly, and a cam assembly and wherein the handle comprises a first through-opening dimensioned to receive and hold the thrust assembly, the cam assembly and the elongated stationary outer tube.

4. The drill system of claim 3, wherein the thrust assembly comprises a thrust shaft, and wherein the thrust shaft is configured to slide within the first through-opening of the handle.

5. The drill system of claim 4, wherein the thrust assembly further comprises a shank chuck, a thrust end cap, and first and second thrust bushings.

6. The drill system of claim 4, wherein the drill bit guidance device further comprises a drive shaft, and a drill bit and wherein said actuating tube surrounds said drive shaft and wherein said drill bit is attached to a distal end of the drive shaft and a proximal end of the drive shaft is attached to the thrust shaft.

7. The drill system of claim 6, wherein proximal ends of the actuating tube, the drive shaft and the actuator tube are linked together via the cam assembly, and wherein the cam assembly is configured to maintain constant the ratio of the length of the drive shaft to the length of the collapsed slotted distal end portion of the actuating tube, thereby maintaining constant a radius of the curved path.

8. The drill system of claim 3, wherein the cam assembly comprises a central cam and a distal cam and wherein the central cam and distal cam are configured to turn and slide within the first through-opening of the handle.

9. The drill system of claim 4, wherein the radius of the curved path is configured to remain constant by maintaining constant the ratio of the length of the flexible drive cable to the length of the collapsed slotted distal end portion of the elongated actuating tube.

10. The drill system of claim 6, wherein the drive shaft comprises a flexible cable and wherein the flexible cable comprises Nitinol wire or stainless steel wire.

11. The drill system of claim 1, wherein each of said slots in the actuating tube is wider at the bottom than at the top.

12. The drill system of claim 1, further comprising a drill driver comprising a front opening configured to receive removable attachments, and wherein the drill bit guidance device is configured to be removably attached to the front opening of the drill driver.

13. A method for forming a curved tunnel in a bone comprising:
providing a drill and a drill bit guidance device comprising an elongated stationary outer tube extending along a first axis and an elongated inner tube assembly and wherein said elongated inner tube assembly is configured to slidably move within the elongated stationary outer tube along the first axis and to exit from a distal end of the elongated outer tube;
wherein said elongated inner tube assembly comprises an actuating tube and an actuator tube, wherein the actuating tube is configured to slidably move within the actuator tube and comprises a plurality of semi-cross-sectional slots extending in a slot direction perpendicular to the first axis and wherein each slot of the actuating tube is configured to collapse inward in the slot direction when exiting the distal end of the elongated outer tube, thereby causing the distal end portion of the elongated inner tube assembly to follow a curved path.

14. The method of claim 13, wherein a distal end of the actuator tube is connected to a distal end of the actuating tube, and wherein the actuator tube comprises a partially slotted distal end portion and wherein the slots of the actuating tube are displaced by 180 degrees relative to slots of the actuator tube.

15. The method of claim 13, wherein the drill bit guidance device further comprises a handle, a thrust assembly, and a cam assembly and wherein the handle comprises a first through-opening dimensioned to receive and hold the thrust assembly, the cam assembly and the elongated stationary outer tube.

16. The method of claim 15, wherein the thrust assembly comprises a thrust shaft, and wherein the thrust shaft is configured to slide within the first through-opening of the handle.

17. The method of claim 16, wherein the thrust assembly further comprises a shank chuck, a thrust end cap, and first and second thrust bushings.

18. The method of claim 16, wherein the drill bit guidance device further comprises a drive shaft, and a drill bit and wherein said actuating tube surrounds said drive shaft and wherein said drill bit is attached to a distal end of the drive shaft and a proximal end of the drive shaft is attached to the thrust shaft.

19. The method of claim 18, wherein proximal ends of the actuating tube, the drive shaft and the actuator tube are linked together via the cam assembly, and wherein the cam assembly is configured to maintain constant the ratio of the length of the drive shaft to the length of the collapsed slotted distal end portion of the actuating tube, thereby maintaining constant a radius of the curved path.

20. The method of claim 15, wherein the cam assembly comprises a central cam and a distal cam and wherein the central cam and distal cam are configured to turn and slide within the first through-opening of the handle.

21. The method of claim 16, wherein the radius of the curved path is configured to remain constant by maintaining constant the ratio of the length of the flexible drive cable to the length of the collapsed slotted distal end portion of the elongated actuating tube.
